(19) United States
(12) Patent Application Publication
(10) Pub. No.: US 2022/0273165 A1
(43) Pub. Date: Sep. 1, 2022

(54) PORTABLE AND ERGONOMIC ENDOSCOPE WITH DISPOSABLE CANNULA

(71) Applicant: MICRONVISION CORP, Bellevue, WA (US)

(72) Inventor: Xiaolong OUYANG, Bellevue, WA (US)

(21) Appl. No.: 17/745,526

(22) Filed: May 16, 2022

Related U.S. Application Data
(63) Continuation-in-part of application No. 17/473,587, filed on Sep. 13, 2021, now Pat. No. 11,330,973.
Application No. 17/473,587 is a continuation-in-part of application No. 17/362,043, filed on Jun. 29, 2021, now Pat. No. 11,350,816.
Application No. 17/362,043 is a continuation-in-part of application No. PCT/US2019/036060, filed on Jun. 7, 2019.
Application No. PCT/US2019/036060 is a continuation-in-part of application No. 16/363,209, filed on Mar. 25, 2019.
Application No. 16/363,209 is a continuation-in-part of application No. PCT/US2017/053171, filed on Sep. 25, 2017.
(60) Provisional application No. 63/218,362, filed on Jul. 4, 2021.
(60) Provisional application No. 63/213,499, filed on Jun. 22, 2021.
(60) Provisional application No. 63/210,034, filed on Jun. 13, 2021.
(60) Provisional application No. 63/197,639, filed on Jun. 7, 2021.
(60) Provisional application No. 63/197,611, filed on Jun. 7, 2021.
(60) Provisional application No. 63/183,151, filed on May 3, 2021.
(60) Provisional application No. 63/153,252, filed on Feb. 24, 2021.
(60) Provisional application No. 63/149,338, filed on Feb. 14, 2021.
(60) Provisional application No. 63/138,751, filed on Jan. 18, 2021.

(51) Int. Cl. A61B 1/00; A61B 1/06; A61B 1/04; A61B 1/015; A61B 1/005; A61B 1/05

(52) U.S. Cl. CPC A61B 1/00103; A61B 1/0646; A61B 1/0655; A61B 1/043; A61B 1/00009; A61B 1/0005; A61B 1/015; A61B 1/0051; A61B 1/05; A61B 1/00105; A61B 1/00066; A61B 1/00052; A61B 1/00108

(57) ABSTRACT

A multi-camera, multi-spectral endoscope provides a composite image formed from a white light stereo image and a fluorescence image. The fluorescence image highlights areas of abnormal tissue without obscuring the white light image. In one example, the endoscope uses a white light camera and a camera that has an electrically controlled color filter that switches between passing white light and passing fluorescent light. In another example, two white light cameras produce a stereo image, and a third camera is a dedicated fluorescence camera. In yet another example, one pair of cameras generates a white light stereo image, and another pair generated a stereo fluorescence image. The endoscope can use a single-use portion comprising the cameras and a reusable portion and can rotate the cannula and bend it distal portion. In another example, one of the single-use portion and the reusable portion has an axial slot and the other has an axial rail that slides in the slot in one direction to assemble the endoscope and in another to separate the two portions.

### REFERENCE TO RELATED APPLICATIONS

This application is a continuation-in-part of U.S. patent application Ser. No. 17/473,587 filed Sep. 13, 2021 and now U.S. Pat. No. 11,330,973, which is a continuation-in-part of each of: U.S. patent application Ser. No. 17/362,043 filed Jun. 29, 2021 and allowed on Apr. 13, 2022; International Patent Appl. No. PCT/US19/36060 filed Jun. 7, 2019; U.S. patent application Ser. No. 16/363,209 filed Mar. 25, 2019 and published as US Pat. Appl. Publ. No. US2019/0216325, and International Patent Appl. No. PCT/US17/53171 filed Sep. 25, 2017.

This application incorporates by reference the entirety of the foregoing patent applications and claims the benefit of the filing date of each of the above-identified patent applications, as well as of the applications that they incorporated by reference, directly or indirectly, and the benefit of which they claim, including U.S. provisional applications, U.S. non-provisional applications, and International applications.

Said U.S. Patent Appl. Ser. No. 17,473,587 claims the benefit of and incorporates by reference each of the following provisional applications:
U.S. Prov. Ser. No. 63/218,362 filed Jul. 4, 2021
U.S. Prov. Ser. No. 63/213,499 filed Jun. 22, 2021
U.S. Prov. Ser. No. 63/210,034 filed Jun. 13, 2021
U.S. Prov. Ser. No. 63/197,639 filed Jun. 7, 2021
U.S. Prov. Ser. No. 63/197,611 filed Jun. 7, 2021
U.S. Prov. Ser. No. 63/183,151 filed May 3, 2021;
U.S. Prov. Ser. No. 63/153,252 filed Feb. 24, 2021;
U.S. Prov. Ser. No. 63/149,338 filed Feb. 14, 2021;
U.S. Prov. Ser. No. 63/138,751 filed Jan. 18, 2021;
U.S. Prov. Ser. No. 63/129,703 filed Dec. 23, 2020;
U.S. Prov. Ser. No. 63/124,803 filed Dec. 13, 2020;
U.S. Prov. Ser. No. 63/121,924 filed Dec. 6, 2020;
U.S. Prov. Ser. No. 63/121,246 filed Dec. 4, 2020;
U.S. Prov. Ser. No. 63/107,344 filed Oct. 29, 2020;
U.S. Prov. Ser. No. 63/087,935 filed Oct. 6, 2020;
U.S. Prov. Ser. No. 63/083,932 filed Sep. 27, 2020;
U.S. Prov. Ser. No. 63/077,675 filed Sep. 13, 2020; and
U.S. Prov. Ser. No. 63/077,635 filed Sep. 13, 2020.

This patent application is also related to and incorporates by reference each of the following international, non-provisional and provisional applications:
International Patent Application No. PCT/US17/53171 filed Sep. 25, 2017;
U.S. Pat. No. 8,702,594 Issued Apr. 22, 2014;
U.S. patent application Ser. No. 16/363,209 filed Mar. 25, 2019;
International Patent Application No. PCT/US19/36060 filed Jun. 7, 2019;
U.S. patent application Ser. No. 16/972,989 filed Dec. 7, 2020;
U.S. Prov. Ser. No. 62/816,366 filed Mar. 11, 2019;
U.S. Prov. Ser. No. 62/671,445 filed May 15, 2018;
U.S. Prov. Ser. No. 62/654,295 filed Apr. 6, 2018;
U.S. Prov. Ser. No. 62/647,817 filed Mar. 25, 2018;
U.S. Prov. Ser. No. 62/558,818 filed Sep. 14, 2017;
U.S. Prov. Ser. No. 62/550,581 filed Aug. 26, 2017;
U.S. Prov. Ser. No. 62/550,560 filed Aug. 25, 2017;
U.S. Prov. Ser. No. 62/550,188 filed Aug. 25, 2017;
U.S. Prov. Ser. No. 62/502,670 filed May 6, 2017;
U.S. Prov. Ser. No. 62/485,641 filed Apr. 14, 2017;
U.S. Prov. Ser. No. 62/485,454 filed Apr. 14, 2017;
U.S. Prov. Ser. No. 62/429,368 filed Dec. 2, 2016;
U.S. Prov. Ser. No. 62/428,018 filed Nov. 30, 2016;
U.S. Prov. Ser. No. 62/424,381 filed Nov. 18, 2016;
U.S. Prov. Ser. No. 62/423,213 filed Nov. 17, 2016;
U.S. Prov. Ser. No. 62/405,915 filed Oct. 8, 2016;
U.S. Prov. Ser. No. 62/399,712 filed Sep. 26, 2016;
U.S. Prov. Ser. No. 62/399,436 filed Sep. 25, 2016;
U.S. Prov. Ser. No. 62/399,429 filed Sep. 25, 2016;
U.S. Prov. Ser. No. 62/287,901 filed Jan. 28, 2016;
U.S. Prov. Ser. No. 62/279,784 filed Jan. 17, 2016;
U.S. Prov. Ser. No. 62/275,241 filed Jan. 6, 2016;
U.S. Prov. Ser. No. 62/275,222 filed Jan. 5, 2016;
U.S. Prov. Ser. No. 62/259,991 filed Nov. 25, 2015;
U.S. Prov. Ser. No. 62/254,718 filed Nov. 13, 2015;
U.S. Prov. Ser. No. 62/139,754 filed Mar. 29, 2015;
U.S. Prov. Ser. No. 62/120,316 filed Feb. 24, 2015; and
U.S. Prov. Ser. No. 62/119,521 filed Feb. 23, 2015.

All the above-referenced non-provisional, provisional and international patent applications are collectively referenced herein as “the commonly assigned incorporated applications.”

### FIELD

This patent specification generally relates mainly to endoscopes. More particularly, some embodiments relate to portable endoscope devices that include a re-usable handle portion and a disposable or single-use cannula portion.

### BACKGROUND

In the case of both rigid and flexible conventional endoscopes, the lens or fiber optic system is relatively expensive and is intended to be re-used many times. Therefore, stringent decontamination and disinfection procedures need to be carried out after each use. Disposable endoscopy is an emerging category of endoscopic instruments. In some cases, endoscopes can be made at a low enough cost for single-use applications. Disposable or single-use endoscopy lessens the risk of cross-contamination and hospital acquired diseases.

The subject matter described or claimed in this patent specification is not limited to embodiments that solve any specific disadvantages or that operate only in environments such as those described above. Rather, the above background is only provided to illustrate one exemplary technology area where some embodiments described herein may be practiced.

### SUMMARY OF INITIALLY CLAIMED SUBJECT MATTER

According to some embodiments, a multi-camera, multi-spectral endoscope comprises: a cannula configured for insertion in a patient; a forward-looking camera CamW at a distal portion of the cannula views a target and is responsive primarily to a wavelength range of white light; an electrically controlled color filter also at the distal portion of the cannula and configured to selectively operate in a mode A to pass light primarily in a wavelength range of white light or in a mode B to pass to said camera CamF light primarily in a selected narrow wavelength band or fluorescence light; a forward-looking camera CamFA/B also at the distal portion of the cannula views said target from a different angle and through said color electrically controlled filter; a processing system configured to: selectively switch said color filter between mode A and mode B, and receive image data from said cameras CamW and Cam FA/B and form a white light stereo image of the target when said filter is operating in mode A but form a selected narrow wavelength band or fluorescence light image from camera CamFA/B when said filter is operating in mode B; and an image display; wherein said processing system and image display are configured to form and display a composite image as an overlay of the white light stereo image and the selected narrow wavelength band light or fluorescent light image.

According to some embodiments, the multi-camera, multi-spectral endoscope can further include on or more of the following features: (a) a fluid hub from which said cannula extends distally and a hand piece to which the fluid hub us secured; (b). the fluid hub and cannula can comprise a single-use unit and said hand piece can comprises a reusable unit that is releasably secured to the single-use unit; (c). said cannula can be configured to rotate relative to a proximal portion of said fluid hub; (e) the endoscope can include a manual bend controller and said cannula's distal portion can be configured to bend in response to operation of said manual bend control; (f) said camera CamF can have a lower spatial resolution than said camera CamW at least when said filter is operating in said mode B.

According to some embodiments, a multi-camera, multi-spectral endoscope comprises: a tubular cannula configured for insertion in a patient; a first forward-looking camera system located at a distal portion of the cannula and comprising two cameras CamW1 and CamW2 viewing the same target from different angles and responsive primarily to a CamW1 wavelength range and a CamW2 wavelength range respectively; a second camera system located at the distal portion of the cannula and comprising a camera CamF that also views said target but is responsive primarily to a CamF wavelength range that is different from at least one of the CamW1 and CamW2 wavelength ranges; a processing system configured to receive image data from said first and second camera systems and to process the received image data into a stereo image of the target using image data from CamW1 and Cam W2, a two-dimensional (2D) image of the target using image data from Cam F, and a composite image of the target overlaying said stereo and said 2D images; and a display configured to display said composite image.

According to some embodiments, the endoscope described in the immediately preceding paragraph can further include on or more of the following features: (a) said wavelength ranges CamW1 and CamW2 overlap; (b) said wavelength ranges CamW1 and CamW2 are white light ranges; said CamF range is a selected narrow wavelength band range or fluorescence light (c) said 2D image represents target areas that emit fluoresce above a threshold of likely abnormal tissue, thereby highlighting likely abnormal tissue in said composite image; (d) said composite image comprises an overlay in which said 2D image is visible in areas of said 2D image; (e) said camera CamF has a lower spatial resolution than at least one of said cameras CamW1 and CamW2; (f) the endoscope further includes at least one internal channel in said in which said cannula, a fluid hub from which said cannula extends distally and which communicates with said internal channel, wherein said cannula is configured to rotate relative to a proximal portion of said fluid hub; (g) the endoscope further includes a hand piece to which said fluid hub releasably attaches and which houses at least a portion of said processing system; (h) said display is mounted on said hand piece; and (i) the endoscope further includes a manual bend controller and wherein said cannula's distal portion is configured to bend in response to operation of said manual bend control.

According to some embodiments, a multi-camera, multi-spectral endoscope comprises: a cannula configured for insertion in a patient; a first forward-looking camera system at a distal portion of the cannula and comprising a camera CamW1 and a camera CamW2 viewing a target from different angles and responsive primarily to a CamW1 wavelength range and a CamW2 wavelength range respectively; a second forward-looking camera system also located at the distal portion of the cannula and comprising a camera CamF1 and a camera CamF2 viewing said target from different angles and responsive primarily to a CamF1 wavelength range and a CamF2 wavelength range respectively that differ from at least one of said CamW1 and CamW wavelengths; a processing system receiving image data from said first and second camera systems and processing the received image data into a CamW image of the target based on the image data from said cameras CamW1 and CamW2 and a CamF images of the target based on image data from said cameras Cam F1 and CamF2 overlaid in a composite image; and a display configured to displays said composite image.

According to some embodiments, the endoscope described in the immediately preceding paragraph can further include on or more of the following features: (a) said CamW1 and CamW2 wavelength ranges are white light ranges and said CamF1 and CamF2 wavelength ranges are selected wavelength band or fluorescence light ranges; and (b) each of said images CamW and CamF is a stereo image of the target, and said composite image is an overlay in which the images CamW and CamF are spatially registered.

According to some embodiments, an endoscope comprises: an L-shaped handle portion comprising a downwardly extending handle and an axially extending housing; a hub removably secured to a proximal end of the housing and a cannula extending distally from the hub; wherein: one of said housing and hub comprises an axially extending slot that faces down and the other comprises an axially extending rail that faces up and is configured to slide into the slot in the proximal direction and thereby removably secure the hub and cannula to the handle portion; said hub and said housing comprises respective electrical connectors that mate and make electrical contact when the housing and hub are secured to each other; said proximal portion of the handle portion comprises an opening and said hub and cannula comprise a bending mechanism that is configured to bend a distal portion of the cannula and includes a proximally extending thumb lever that passes through said opening and protrudes distally from the handle portion when the hub and handle portion are secured to each other and manual action on said thumb lever controls bending of said distal portion of the cannula; a camera module at the distal portion of the cannula; and a display operatively coupled with the camera module to receive image data therefrom and display images based thereon.

According to some embodiments, the endoscope described in the immediately preceding paragraph can further include on or more of the following features: (a) the bending mechanism comprises a wheel mounted in said housing for rotation and coupled with said bending lever to rotate in response to manipulation of the bending lever and cables coupled with the wheel and to the distant portion of the cannula to translate rotation of the wheel to bending of said distal portion of the cannula; (b) said hub and cannula separate from the handle portion by manual sliding of the hub in the distal direction relative to the handle portion; and (c) the endoscope includes a lock pin in one of the housing and hub and a catch in the other, configured to engage when the endoscope is assembled and hold the hub to the housing, and a manually operated release to disengage the lock pin and catch from each other to thereby allow removal of the hub from the housing.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail through the use of the accompanying drawings in which:

FIGS. 1A, 1B and 1C are side, top and rear views of a portable and ergonomic endoscope with disposable cannula, according to some embodiments;

FIGS. 2A and 2B are perspective views of a portable and ergonomic endoscope with disposable cannula, according to some embodiments;

FIGS. 3A-3B are perspective views that illustrate the mating and un-mating of reusable and disposable portions of a portable and ergonomic endoscope, according to some embodiments;

FIGS. 4A and 4B are a perspective and a schematic view of a distal tip including multiple camera and lighting modules used with a portable and ergonomic endoscope, according to some embodiments;

FIG. 5 is a schematic diagram of a dual camera dual light source system for multi-spectral imaging and surgical applications, according to some embodiments;

FIG. 6 is a conceptual diagram illustrating design aspects of a dual camera dual light source system for multi-spectral imaging and surgical applications, according to some embodiments;

FIG. 7 is a diagram illustrating possible color filter array configurations for a dual camera dual light source system for multi-spectral imaging and surgical applications, according to some embodiments;

FIG. 8 is a plot showing quantum efficiency versus wavelength for Nyxel and conventional pixels;

FIG. 9 is a diagram illustrating further aspects of combining multi-band image data from a dual camera dual light source system, according to some embodiments;

FIG. 10 is a perspective view in which a combined, spatially registered image displayed to a user on an endoscopy system, according to some embodiments; and

FIG. 11 is a perspective view of a endoscopy system having one or more forward facing cameras, according to some embodiments.

FIG. 12 is a schematic diagram illustrating an endoscope using a camera with an electrically controlled color filter to expose the camera to either white light or fluorescence and another camera doe white light, according to some embodiments.

FIG. 13 is a plan view of a distal end of a cannula using a pair of white light camera and a camera for a selected narrow wavelength band or fluorescence light, and light sources and internal channels in the cannula, according to some embodiments.

FIG. 14 is otherwise like FIG. 13 but shows a different arrangement of the cameras and light sources, and a single internal channel, according to some embodiments.

FIG. 15 is a plan view of a distal end of a cannula using a pair of white light camera and a pair of cameras for a selected narrow wavelength band or fluorescence light, and light sources and internal channels in the cannula, according to some embodiments.

FIG. 16 is otherwise like FIG. 15 but shows a different arrangement of the cameras and light sources, and a single internal channel, according to some embodiments.

FIG. 17 is a perspective view of an endoscope, according to some embodiments.

FIG. 18 is an exploded perspective view of an endoscope, according to some embodiments.

FIG. 19 is an exploded perspective view of portions of an endoscope, according to some embodiments.

FIG. 20 is a sectional view of portions of an endoscope, according to some embodiments.

FIG. 21 is an exploded view of components of an endoscope, according to some embodiments.

FIG. 22 is a top view of an endoscope, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all of these details. Moreover, for the purpose of clarity, certain technical material that is known in the related art has not been described in detail to avoid unnecessarily obscuring the new subject matter described herein. It should be clear that individual features of one or several of the specific embodiments described herein can be used in combination with features of other described embodiments or with other features. Further, like reference numbers and designations in the various drawings indicate like elements.

According to some embodiments, a portable ergonomic endoscope system is described that includes an imaging system with at least two separate cameras and two separate light sources. The camera and light sources are configured to be used to simultaneously image a target object (e.g., tissue). By employing different illuminations, different filters and manipulating the spectral responses, different characteristics of the target object can be captured. According to some embodiments, a system processor can coordinate the cameras, the light sources and combine the resulting images to display to an operator an enhanced combined image of the object. According to some embodiments, the system can be configured to perform NBI (Narrow Band Imaging) imaging. According to some embodiments, the system can also be configured to perform Fluorescence Imaging.

As used herein, the term Color Filter Array (CFA) refers to a filter placed on top of a pixel to allow a certain bandwidth(s) to pass. Regular consumer cameras such as the cell phone camera uses RGB CFA. For other special applications, special CFAs can be designed.

As used herein, the term Narrow-band imaging (NBI) refers to a color imaging technique for endoscopic diagnostic medical tests, where light of specific blue and green wavelengths is used to enhance the detail of certain aspects of the surface of the mucosa. According to some embodiments, a special filter can be electronically activated by a switch in the endoscope leading to the use of ambient light preferably of wavelengths at or close to 415 nm (blue) and 540 nm (green). Because the peak light absorption of hemoglobin occurs at these wavelengths, blood vessels will appear very dark, allowing for their improved visibility and for the improved identification of other surface structures.

As used herein, the term Fluorescence Imaging (FI) refers to fluorescence imaging, sometimes using fluorescent dyes, to mark, highlight or enhance certain biological mechanisms and/or structures. Fluorescence itself, is a form of luminescence that results from matter emitting light of a certain wavelength after absorbing electromagnetic radiation. In a selected narrow wavelength band light endoscopy, for example, fluorescent dyes (Hexvix) are injected in the bladder. Then a selected narrow wavelength band light (around 405 nm) is used to illuminate the tissue with Hexvix which emits fluorescence of wavelength of about 610 nm. Note that with FI, the camera visualizes the fluorescence emitted from within the object, while with NBI the camera visualizes the reflections of various bandwidths of light by the object.

According to some embodiments, a novel dual camera and dual light source (DCDL) system is described for multi-spectral or multi-color imaging. Embodiments of surgical applications are disclosed with simultaneous white light, fluorescence and infrared images.

The described methodologies apply to general multi-spectral multi-band imaging. According to some embodiments, an endoscopy system is described that includes two separate camera/LED systems that are integrated into the same cannula or endoscope. A white light camera, referred to as CamW, is paired with white light LED, referred to as LightW. A fluorescence camera, referred to as CamF is paired with a selected narrow wavelength band light LEDs, referred to as LightC. In this configuration, CamF is used as IR Camera when either or both LightC, LightW are off.

According to some embodiments, CamW is optimized for white light endoscopy, where strong and optimal white LEDs are used to illuminate the object, such that high image resolution can be achieved. CamF is optimized for sensitivity, because typically a fluorescence light source is weak. To maximize sensitivity and signal to noise of the CMOS sensor pixels for high quality imaging, the following are implemented:

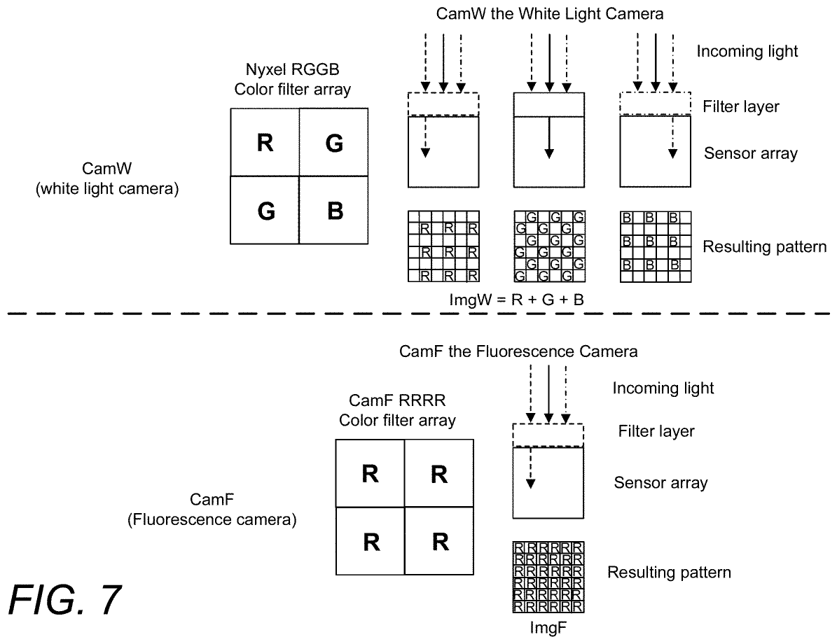

According to some embodiments, a special color filter array (CFA) on the pixel array is used (shown in FIG. 7), such that the CMOS sensor array is sensitive to red or IR spectrum (near 600 nm or higher). According to some embodiments, to further improve sensitivity, preferably relatively large pixels (for example 2.2 um×2.2 um) are used for the CMOS sensor of CamF. In such cases, CamF preferably has lower spatial resolution than CamW pixels (for example, 1.75 um×1.75 um or 1.0 um×1.0 um) but much higher sensitivity.

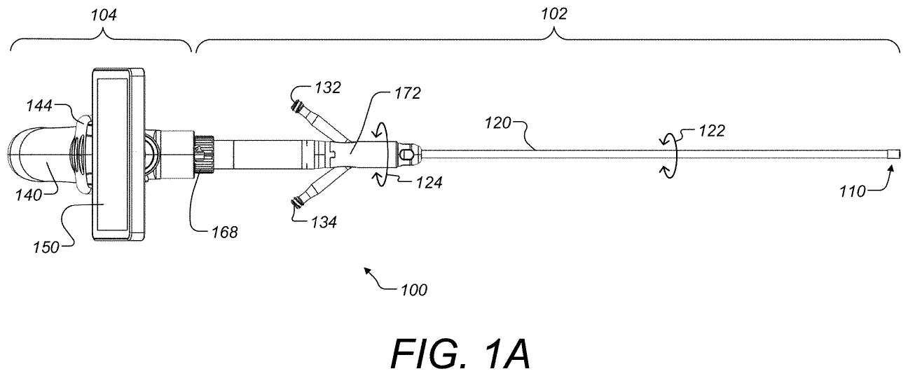

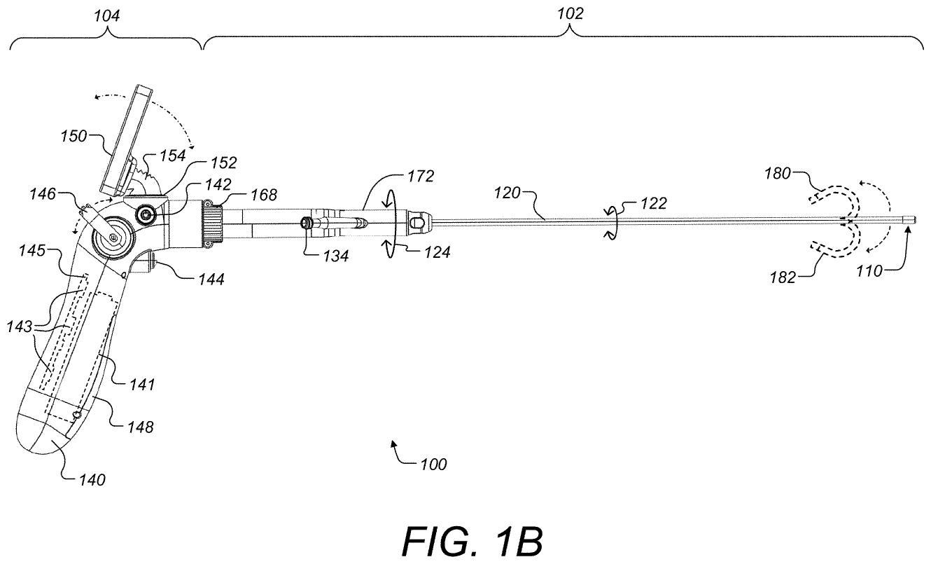

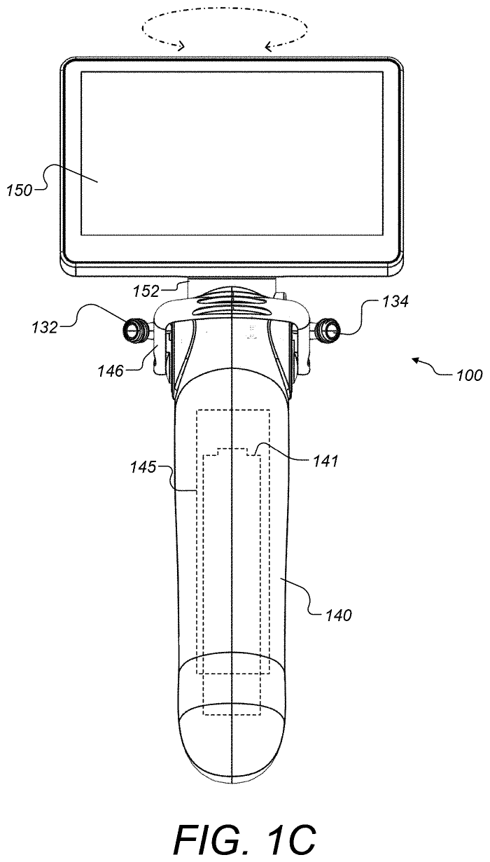

FIGS. 1A, 1B and 1C are side, top and rear views of a portable and ergonomic endoscope with disposable cannula, according to some embodiments. System 100 is adapted for easy and quick use with minimized patient discomfort and high placement accuracy. System 100 is made up of a disposable, or single-use portion 102 and a re-usable portion 104. The two portions 102 and 104 can be mated and un-mated with each other via connectors as will be shown and discussed infra in further detail. Cannula 120 has an imaging and illumination modules on its distal tip 110. An electrical cable (not shown) is positioned within the cannula and supplies control signals and power to the camera and LED illumination modules on distal tip 110 and also transmits video image data from the camera module to the hand piece 140 and display 150 for viewing by an operator. In the example shown, hand piece 140 includes two control buttons 142 and 144 which can be configured for power on/off and image capture, respectively. According to some embodiments, hand piece 140 is shaped as a pistol grip as shown and includes a rechargeable battery 141 that is accessible via battery door 148. According to some embodiments, battery 141 is a lithium-ion rechargeable battery such as type 18650 or 26650. Also housed within handle 140 are electronics modules 143 mounted on printed circuit board (PCB) 145. Electronics modules 143 and PCB 145 are configured to carry out various processes such as video processing and capture, wi-fi transmission of data to external devices, lighting control, user interface processing, and diagnostics. Electronic modules 143 also are configured to include at least one non-volatile memory module for storing captured video and images from the camera module. According to some embodiments, display 150 can both tilt and swivel to provide optimal viewing angle for the operator. Swivel joint 152 is configured to provide swiveling of display 150 as shown by the dash dot arrow in FIG. 10, and hinge joint 154 is configured to provide tilting of display 150 as shown by the dash dot arrows in FIG. 1B. According to some embodiments, the hinge joint is configured to allow for tilting of display in the distal direction of about 90 degrees, or nearly 90 degrees. Such tilting can be useful, for example, when give the operator an unobstructed or less obstructed view. Handle 140 also includes a thumb lever 146 that can be moved upwards or downwards as shown by the dashed arrows. Moving the thumb lever 146 upwards and downwards causes the distal tip 110 to bend upwards and downwards, respectively, as shown by dashed outlines 180 and 182, respectively. Further details of the operation of thumb lever 146 to control the steering of distal tip 110 and canula 120 is provided in U.S. patent application Ser. No. 17/362,043 filed Jun. 29, 2021, incorporated by reference herein, which is referred to herein as “the '043 Application.”

The cannula 120 is connected proximally to a fluid hub 172 including in this example two fluid ports 132 and 134. Proximal to the fluid hub is a collar 168. According to some embodiments, the collar 168 is configured to rotate so as to allow for a “plug and twist lock” style mating of portions 102 and 104, as will be shown and described in further detail infra. According to some embodiments, at least a portion of fluid hub 172, along with cannula 120 and distal tip 110, are manually rotatable relative to handle 140 along the main longitudinal axis of cannula 120, as shown by solid arrow 124. Thus, rotating the rotatable portion of hub 172 causes rotation of cannula 120 and distal tip 110 as shown by solid arrow 122. According to some embodiments, the combination of rotating canula 120 and 110 and moving the thumb lever 146, the user can “steer” the direction of distal tip 110 as desired. According to some embodiments, the cannula 120 has a preferred working length of roughly 12 inches but shorter or longer lengths can be used depending on the medical application, and a preferred outer diameter of 5.5 to 6.5 inches but again a greater or a lesser diameter can her used depending on the medical application and developments in camera and illumination technology.

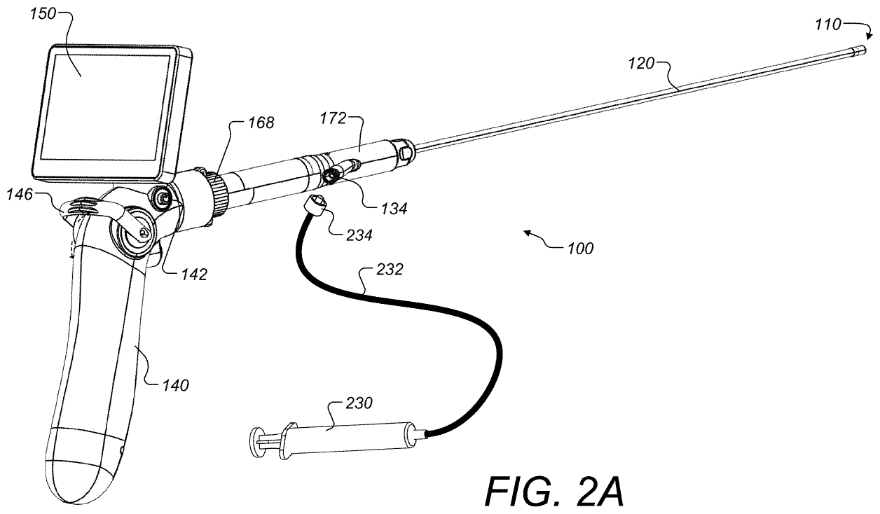

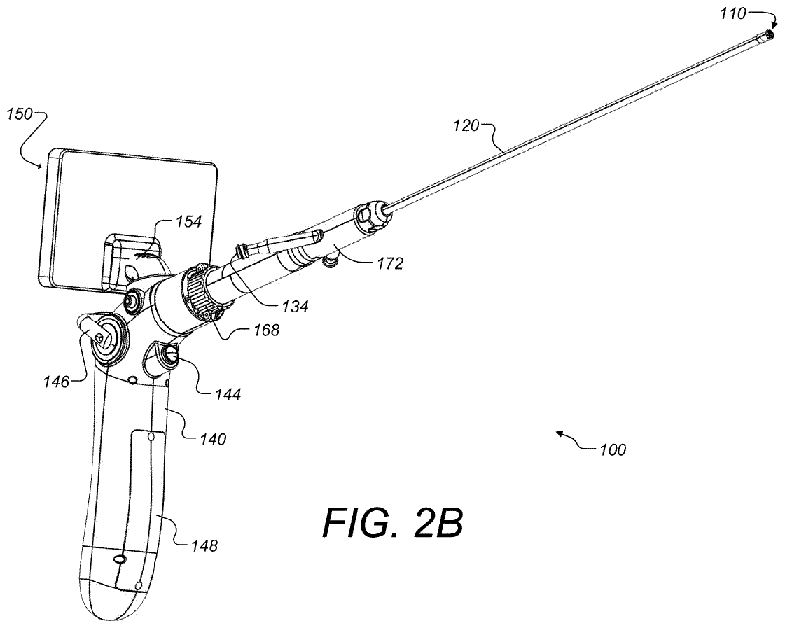

FIGS. 2A and 2B are perspective views of a portable and ergonomic endoscope with disposable cannula, according to some embodiments. FIG. 2A shows a syringe 230 used to supply fluid, such as saline, through a fluid lumen (not shown) within cannula 120 via tubing 232, connector 234 and fluid port 134. According to some embodiments the cannula 120 is semi-rigid. The cannula 120 is stiff enough so it does not collapse with longitudinal pushing and pulling forces expected in a medical procedure for which it is intended. On the other hand, cannula 120 is flexible enough such that it can bend while it passes through curved anatomy.

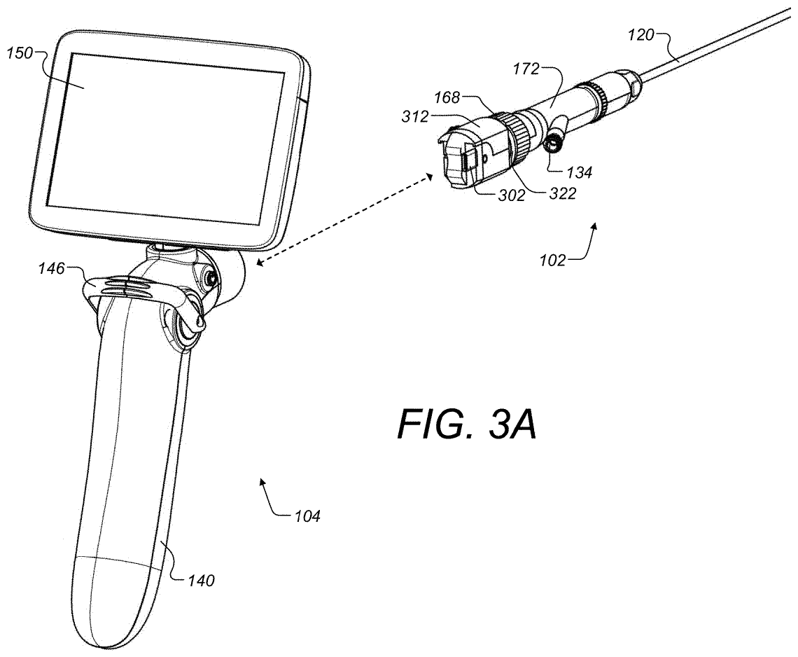

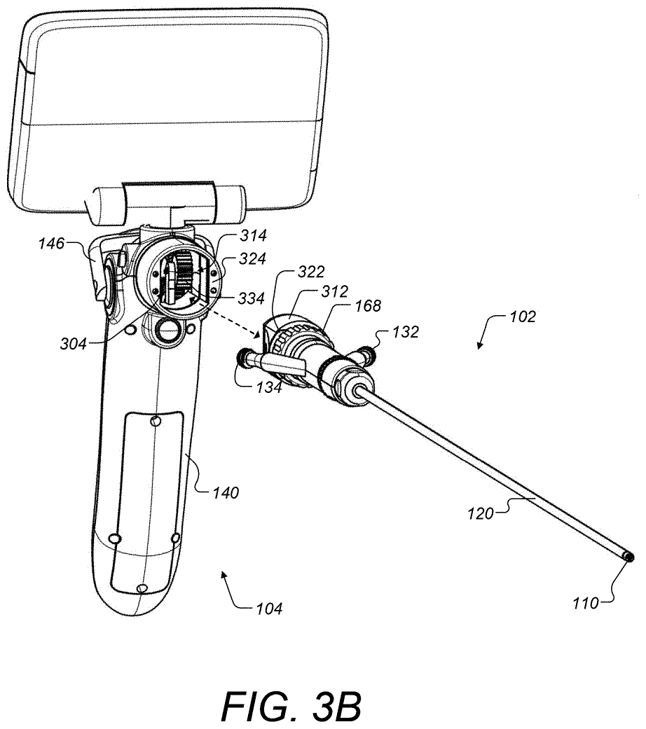

FIGS. 3A-3B are perspective views that illustrate the mating and un-mating of reusable and disposable portions of a portable and ergonomic endoscope, according to some embodiments. The portions 102 and 104 are connectable and separable via a mechanical and electrical connector. The electrical connection is made via a USB-C type plug 302 on single-use portion 102 (visible in FIG. 3A) and USB-C type receptacle 304 on multiple use portion 104 (FIG. 3B). The mechanical connection includes both a structural connection to fixedly attach portions 102 and 104 as well as a steering connection, through which steering input from the steering structure in the re-usable portion 104 can be relayed to the steering components in the single-use portion 102. The structural connection, in this example, includes a male rounded portion 312 on single-use portion 102 that is shaped to fit snugly into a female socket 314 on multiple-use portion 104. The structural connection also includes a twist lock type mechanism wherein a male portion 322 can be inserted past a female opening 324 and then locked by twisting the male portion 322 approximately one quarter turn (90 degrees). The twisting action can be applied manually via textured or knurled ring collar 168. In this way, the connection can be configured as a “plug and twist” type connection. The steering connection is provided by meshing the transmission gear 334 on the multiple-use portion 104 with the passive gear 332 on the single-use portion 102.

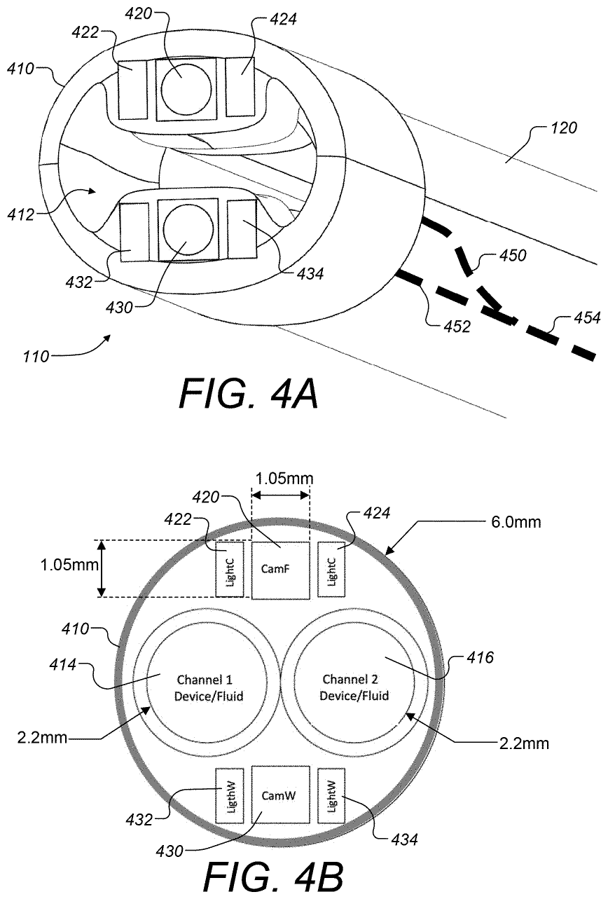

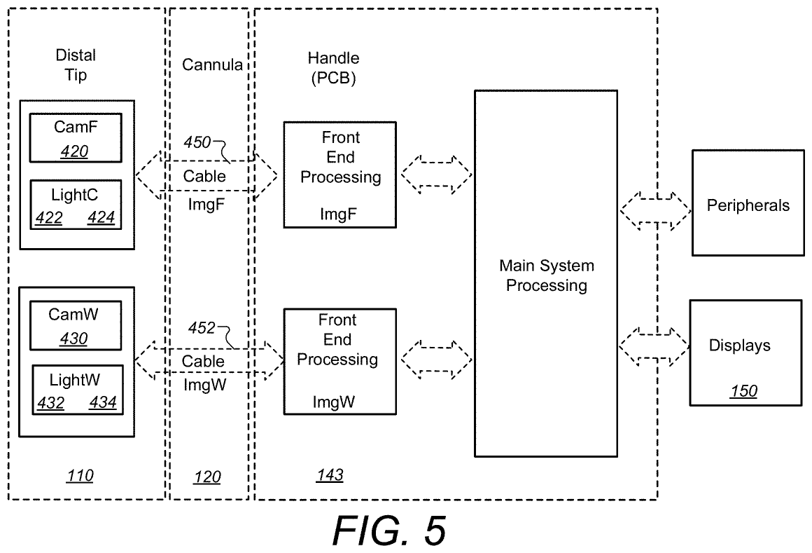

FIGS. 4A and 4B are a perspective and schematic view of a distal tip including multiple camera and lighting modules used with a portable and ergonomic endoscope, according to some embodiments. In FIG. 4A, the distal tip 110 is shown attached to the distal end of canula 120. According to some embodiments, tip 110 includes a housing piece 410 that is molded separately from and bonded to the distal end of canula 120. Housed within housing 410 are two camera modules: CamF module 420 and CamW module 430. Each of the CamF 420 and CamW 430 modules includes a lens and sensor. The sensors for each CamF 420 and CamW 430 include a color sensor, color filter array, and electronics and circuitry as will be described in further detail, infra. On either side of CamF module 420 are two selected narrow wavelength band LEDs 422 and 424 configured to emit excitation light suitable for fluorescence endoscopy. In some examples, LEDs 422 and 424 are configured to emit light at about 410 nm (violet-blue). On either side of CamW module 430 are two white LEDs 430 and 434 configured to emit white light suitable for visible white light endoscopy. Also shown in FIG. 4A is port 412 that is configured to provide fluid (flowing either into or out of the patient) and/or provide an opening through which a tool or other device can pass (e.g., a needle). Note that although FIG. 4A shows a total of four LEDs (two white and two selected narrow wavelength band), in general, other numbers of LEDs may be provided according to factors such as desired lighting quality, endoscope size, and LED characteristics such as size and brightness. In some embodiments three or fewer LEDs can be provided and in some embodiments 10 or more LEDs can be provided. Furthermore, the number of white and wavelength band LEDs does not have to be equal, but also will depend on various factors. The LED set can be 3, 4 or more. Other light sources can be substituted, such as optic fibers that deliver light generated elsewhere.

In FIG. 4B, the example shown includes two separate device/fluid channels 414 and 416. In this case, both have an inner diameter of 2.2 mm. According to some embodiments, channel 414 can be connected to fluid port 134 (shown in FIG. 1A) while channel 416 is connected to fluid port 132 (also shown in FIG. 1A). According to some embodiments, to boost sensitivity to fluorescence the CMOS sensor of CamF 420 is configured with larger pixels than CamW 430. For example, the CamF pixels can be 2.2 um×2.2 um arranged in a 400×400 matrix size, while the CamW pixels are 1.0 um×1.0 um or 1.75 um×1.75 um and arranged in higher spatial resolution matrix size. Because white LEDs tend to be relatively strong, the CamW 430 module can include a CMOS sensor with smaller pixels, such as 1.75 um×1.75 um or 1 um×1 um, so higher spatial resolutions can be achieved with up to 720×720 matrix size or larger.

According to some embodiments, CamF 420 is used for a selected narrow wavelength band light endoscopy, with partial CFA. An example is shown in FIG. 7 where only R Filters are used so that blue light and green light are filtered out and the majority of light that reaches the sensor is red. According to some embodiments, an IR camera is used as CamF.

FIG. 5 is a schematic diagram of a dual camera dual light source system for multi-spectral imaging and surgical applications, according to some embodiments. As shown the distal tip 110 includes the camera and lighting modules, namely CamF, LightC, CamW and LightW. CamF camera 420 is configured for capturing images of a particular color or bandwidth, such as fluorescence with a narrow band centered around 610 nm. Filters for CamF 420 are designed to block incoming light at other wavelengths, for example by using a specially designed CFA array. CamF can be used for either NBI or FI depending on the particular application. LightC light source (422 and 424) for CamF 420, can be the excitation light in case of fluorescence imaging or simply blue or green light in the case of NBI. LEDs or special light sources can be used. According to some embodiments, CamW 430 is regular white light camera such as the camera of a cell phone. A typical RGB CFA can be used and in addition an IR filter can also be used. Typically, an IR filter that filters out 50% of wavelength above 650 nm can be used. LightW (432 and 434), the light source for CamW, can be LED lights with various color tones close to white day light. The cannula 120 includes cables 450 and 452. ImgF refers to the image captured by CamF, and may be fluorescence or, in the case of NBI, reflections of green or blue lights. ImgW refers to the image captured by CamW, which maybe fluorescence or, in the case of NBI, reflections of green or blue lights.

Because the endoscope has two cameras that can operate at the same time and with different combination of lighting such as LightC, LightW (or another light band) the system takes advantage of having two “eyes” looking at the same target but seeing different aspects of the target at the same time and thus extracting more information from or about the objet and targets. For example, when blue light is on, CamF would see mostly fluorescent emission by CamW sees at the same time reflection (that can be very strong) of LightC from the object and a little bit of fluorescence. As the two cameras are in sync and also spatially registered relative to each other, composite information of different kinds is delivered to the user to improve the clinical experience over the case of seeing only one of the two kinds of information about the object or target.

According to some embodiments, Nyxel technology can be used which has been developed by OmniVision. Nyxel pixels can be used for CamF 420 and have significantly improved pixel sensitivity especially with sensitivity to red and near infrared bandwidth. This is particularly useful for detecting fluorescence around 610 nm.

In electronics modules 143, front end processing and main system processing is performed. According to some embodiments, the images are combined for display on display 150.

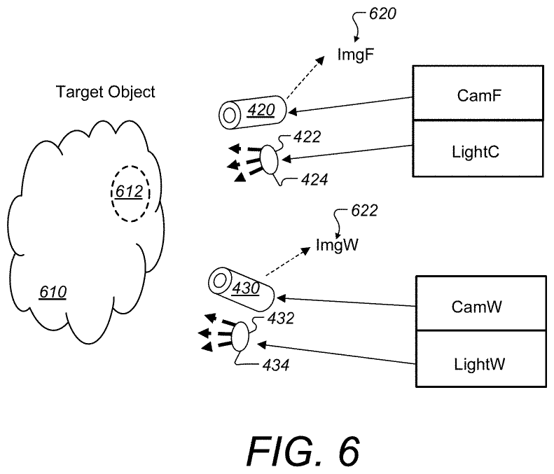

FIG. 6 is a conceptual diagram illustrating design aspects of a dual camera dual light source system for multi-spectral imaging and surgical applications, according to some embodiments. In general, it is desirable to obtain multi-color or multi-spectral images of target objects such as human tissue. Typically, visible light images of the object plus images obtained by other color bands are used to better characterize the target tissue and shape. Two cameras (Cam F, CamVV) are associated two light sources (LightC, LightW). CamF is an optical camera that is sensitive to certain color band, for example Red and IR. The output of CamF is ImgF. LightC is a light source (band C), other than white light. In Dual Band Imaging (DBI), LightC can be green or blue. In fluorescence imaging it can also be a light source that excites the object to fluorescence color. CamW is an optical camera that is sensitive to certain color band (B), for example the white light. The output of CamW is ImgW. LightW is a light source that emits certain color band B, for example the white light.

FIG. 7 is a diagram illustrating possible color filter array configurations for a dual camera dual light source system for multi-spectral imaging and surgical applications, according to some embodiments. According to some embodiments, CamF uses a Nyxel pixel (from Omnivision) and a “Red-Only” filter array, the CamF RRRR filter. This arrangement allows for red and/or IR band to pass while filtering out the background blue and green light.

The CamF can achieve four times the resolution for red compared to that of Nyxel CFA or Old CFA, because one out of four pixels in Nyxel or Old CFA arrangements are used to capture red color. On the other hand, every pixel in CamF arrangement in FIG. 7 is used to capture red color.

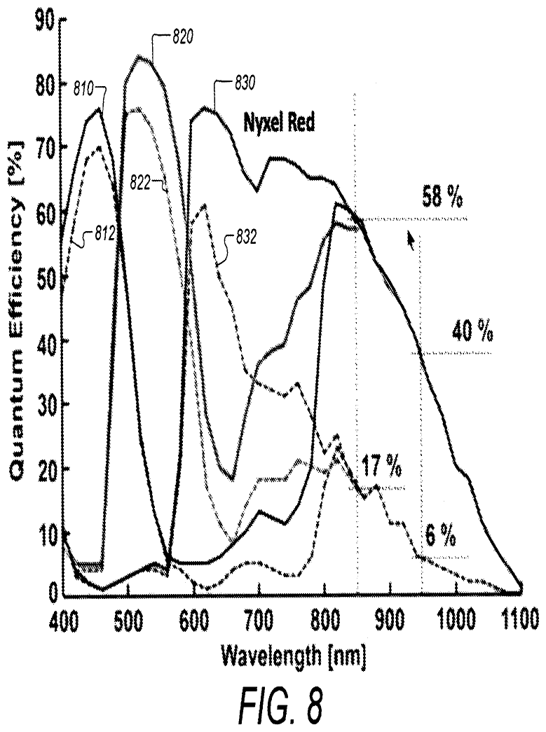

FIG. 8 is a plot showing quantum efficiency versus wavelength for Nyxel and conventional pixels. In this figure, quantum efficiency is shown the new sensor developed by OminiVision, the Nyxel pixel. Curve 810 is a Nyxel blue pixel. Curve 812 is a conventional blue pixel. Curve 820 is a Nyxel green pixel. Curve 822 is a conventions green pixel. Curve 830 is a Nyxel red pixel. Curve 832 is conventional red pixel. It can be seen especially curves 830 and 832 that the Nyxel red pixel has a significantly higher sensitivity to the red or IR band than the regular conventional red pixel.

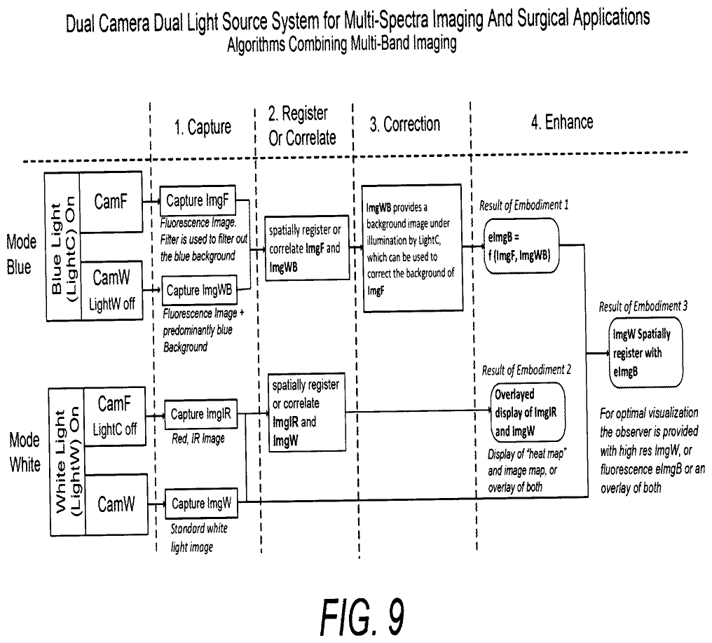

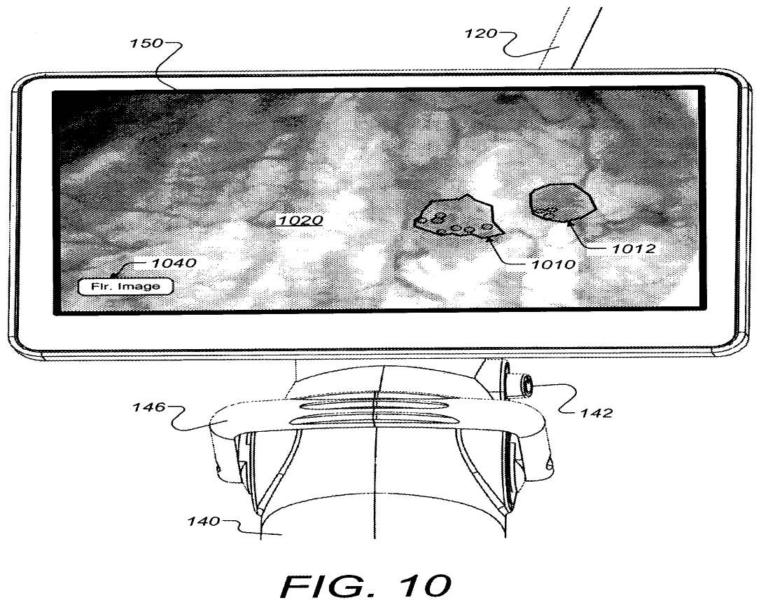

FIG. 9 is a diagram illustrating further aspects of combining multi-band image data from a dual camera dual light source system, according to some embodiments. With the availability of global shutter capability CamF, CamW can capture image frames under different combinations of LightC and LightW being turned “on” or “off.” In “Surgical Embodiment 1” with the LightC (blue light) “on” but the LightW “off”, the resulting captured images are ImgF from CamF and ImgWB from CamW. ImgF and ImgWB are spatially registered or correlated. This can be done due to the short time lag (or completely in sync when both cameras capture simultaneously) between images captured by the different cameras. ImgWB provides a background image under illumination by LightC, which can be used to correct the background of ImgF. The ImgF data combined with ImgWB when only LightC is on produces “eImgB.”

In the case of Blue Light Endoscopy, ImgF has low signal to noise ratio (due to weak fluorescence signal), therefore CMOS sensor with high signal to noise pixels is used. On the other hand, ImgW has high signal to noise (due to strong white light), therefore CMOS sensor with smaller pixels can be used to boost spatial resolution.

In “Surgical Embodiment 2” CamF is used to capture ImgIR with the LightC “off.” CamW captures the standard white light image with LightW “on.” In this case ImgIR provides a “heat map” of the target; it is useful when energy devices such as laser or RF are used for tissue modification. ImgIR can alert users of hot or cold spots. The ImgIR and ImgW data can be spatially registered or correlated, again, due to the short time lag (or no time lag) between images captured by the different cameras. ImgIR and ImgW can also be combined or overlayed to provide a precise location of the hot and cold spots. That is, the hot and cold spots can be viewed in the context of an ordinary standard white light image to provide the viewer with locational context of the hot and cold spots.

In “Surgical Embodiment 3” ImgW is combined with eImgB. By combining embodiments 1 and 2, the high quality eImgB data is spatially registered with the white light image ImgW. The observer is provided with high res ImgW, or fluorescence eImgB or an overlay of both. According to some embodiments, surgeons can employ images available to better visualize their targets. The fluorescence Image eImgB, the white light image ImgW and IR Image ImgIR and seamlessly switch between different visualization modes.

According to a fourth “Embodiment 4” (not shown in FIG. 9) with accumulation of clinical cases, artificial intelligence algorithm (or machine learning) can be designed for automated diagnosis.

FIG. 10 is a perspective view in which a combined, spatially registered image is displayed to a user on an endoscopy system, according to some embodiments. In the displayed view, the ordinary white light image (ImgW) 1020 is displayed over most of the display screen 150. The example shown is “Embodiment 3” shown in FIG. 9, where the eImgB image is combined and spatially registered with the standard white color image (ImgW). In this case the regions 1010 and 1012 are obtained from the eImgB data and clearly show cancerous tumors. The operator can easily view the cancerous regions 1010 and 1012 in spatial registration with the ordinary color image of the surrounding tissue. This blending or combination provides a greatly enhanced view of the target tissue. According to some embodiments, the operator can easily switch between different modes (e.g., Embodiment 1, 2 or 3) by pressing a toggle button such as button 142, button 144 (shown in FIGS. 1B and 2B), or by a soft-button 1040 on touch-sensitive display 150.

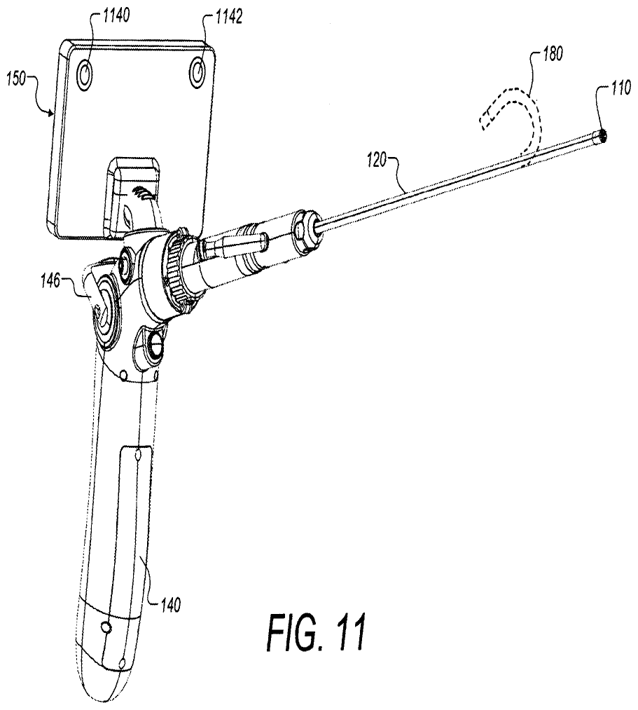

FIG. 11 is a perspective view of an endoscopy system having one or more forward facing cameras, according to some embodiments. The example shown has two forward (distally) facing cameras 1140 and 1142. The forward-facing cameras allow the operator to see precisely where the distal tip is located, without having to move the screen out of the way. During a surgical procedure, especially immediately prior to or during initial insertion of the tip 110, the operator's view can be primary focused on the display screen 150. With forward facing cameras, 1140 and 1142, the precise location of the distal tip and its surroundings can be viewed on the display 150. Image enhancements such as artificially providing a depth of field may be beneficial in some procedures. The two cameras or other means (e.g. LIDAR imaging) may be used to simulate a depth of field centered on the distal tip to enhance usability.

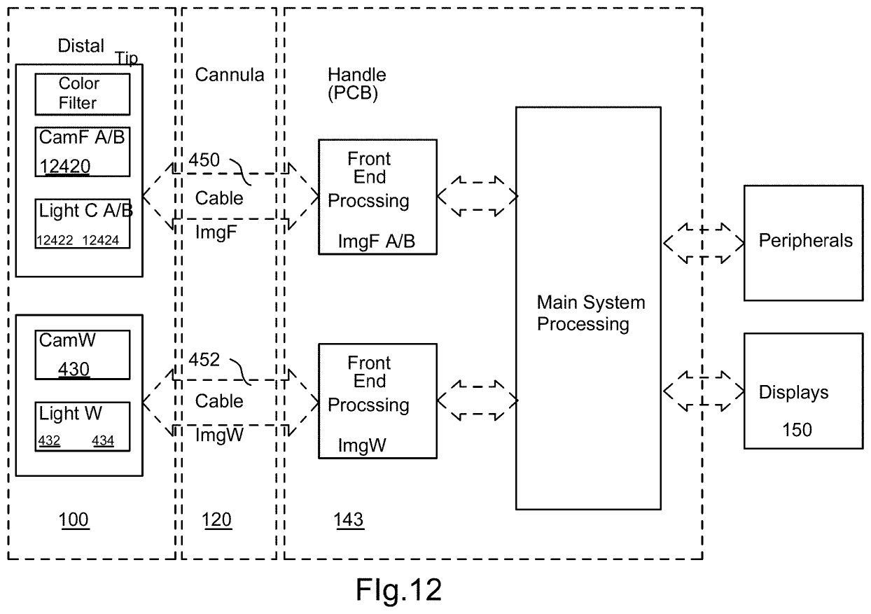

FIG. 12 is like FIG. 5 in other respects but illustrates a multi-camera, multispectral endoscope in which two cameras are used to produce a white light stereo image in one mode of operation but a selected narrow wavelength band light image or a fluorescence image in another mode. The two images can be combined into a composite image like in FIG. 10. In FIG. 12, a forward-looking camera 430 (CamVV) is at a distal portion of cannula 120 to view a target and is responsive primarily to a wavelength range of white light. An electrically controlled color filter 1202 also is at the distal portion of the cannula and is configured to selectively operate in a mode A to pass light primarily in a wavelength range of white light or in a mode B to pass light primarily in a selected narrow wavelength band or fluorescence light. Examples of such filters are discussed in https://en.wikipedia.orG/wiki/Liquid_crystal_tunable_filter and suitable wavelengths and color filters for use in endoscopes are discussed in U.S. application Ser. No. 16/363,209 published as U.S 2019/0216325 A1, both of which are hereby incorporated by reference. A forward-looking camera 12420 (CamFA/B) also at the distal portion of the cannula views said target from a different angle and through said color electrically controlled filter. Cameras 430 and 12420 view a target like the two cameras illustrated in FIG. 6. Processing system 143 configured to selectively switch said color filter between mode A and mode B, and to process image data received from cameras 430 and 12420 to form a white light stereo image of the target when said filter is operating in mode A, but to form a a selected narrow wavelength band image or fluorescence light image from camera CamFA/B when said filter is operating in mode B. An image display 150 displays images and the processing system 143 and display 150 are configured to form and display a composite image as an overlay of the white light stereo image and the selected narrow wavelength band light image or fluorescence light image, like the image illustrates in FIG. 10 in which areas that differ is a selected parameter are highlighted. Processing system 143 can be configured to switch filter 1202 rapidly between modes 1 and 2, for example several times or hundreds of times or more per second, such that the stereo image and the selected narrow wavelength band image or fluorescence image for practical purposes are showing the target essentially in real time. As noted above, the selected narrow wavelength band image or fluorescence light image preferably has lower spatial resolution than the images from the white light cameras. Processing system 143 and display 150 can be configured to selectively display the composite image, or the stereo image, or the selected narrow wavelength band image or fluorescence image, or all three images at the same time. The composite image can be as in FIG. 10—an overlay of two spatially registered images of the same target but taken at different wavelengths of light light.

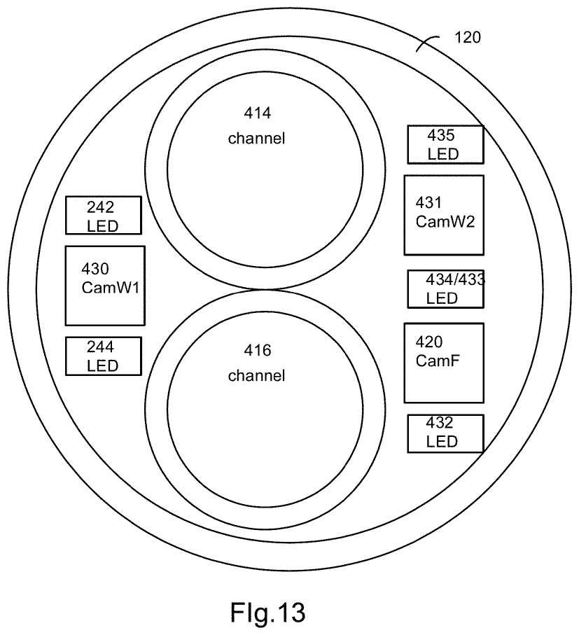

FIG. 13 illustrates a multi-camera, multispectral endoscope in which a first forward-looking camera system provides a white light stereo image of a target, a second camera system provides a selected narrow wavelength band image or fluorescence light image of the target, and a processing system combines the two images into a composite image overlay for display. In FIG. 13, a first forward-looking camera system is located at a distal portion of cannula 120 and comprising two cameras—camera 430 (Cam W1) and camera 431 (CamW2) both viewing the same target but from different angles, like the two cameras in FIG. 6. Camera 430 is responsive primarily to a CamW1 wavelength range and camera 431 is responsive primarily to a CamW2 wavelength range. The two wavelength ranges can be the same white light. A second camera system also is located at the distal portion of cannula 120 and comprises a camera 420 (Cam F) that also views said target but is responsive primarily to a CamF wavelength range that is different from at least one of the CamW1 and CamW2 wavelength ranges. The CamW1 and CmW2 wavelength ranges can be the same white light. The cam wavelength CamF can be a selected narrow wavelength band or fluorescence light. Processing system 143 (FIG. 6) is coupled with the first and second camera system and is configured to receive image data from said first and second camera systems and to process the received image data into a white light stereo image, a selected narrow wavelength band image or fluorescence light image, and a composite image overlaying said stereo image and the selected narrow wavelength band image or fluorescence light image. Processing system 143 also is configured to control LED light sources 242, 244, 432, 434, and 435 to turn them ON or OFF as required for the respective images. In this example, all three cameral in FIG. 13 can view the target concurrently. Processing system 143 and display 150 can be configured to selectively display the composite image, or the stereo image, or the selected narrow wavelength band image or fluorescence image, or all three images at the same time. The composite image can be as in FIG. 10—an overlay of two spatially registered images of the same target but taken at different wavelengths ranges of light. FIG. 13 illustrates two channels in cannula 120-414 and 416— but a single channel or more than two channels can be used.

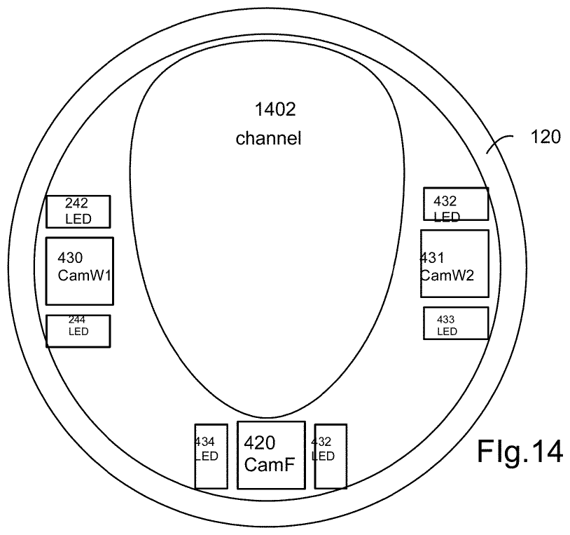

FIG. 14 is otherwise like FIG. 13 but illustrates a multi-camera, multispectral endoscope in which the three cameras and their light sources are arranged differently and cannula 120 has a single channel 1402

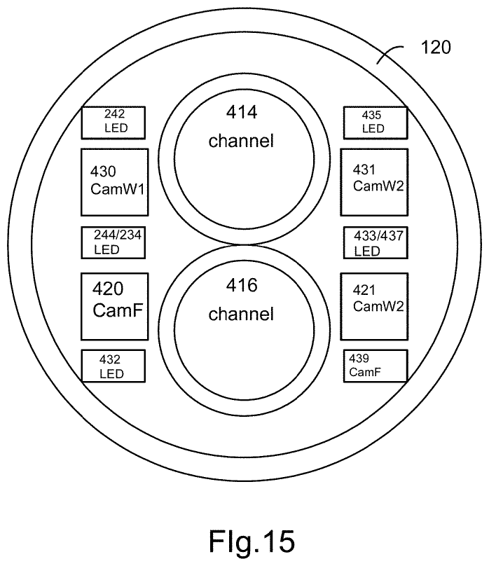

FIG. 15 is otherwise like FIG. 13 but illustrates a multi-camera, multispectral endoscope in which a second forward-looking camera system comprises CamF1 and CanF2, both imaging in the selected narrow wavelength band or fluorescence wavelength ranges such that the system can generate a stereo image both at white light and at the selected narrow wavelength band light or fluorescence light. In FIG. 14, a first forward-looking camera system at a distal portion of the cannula comprises a camera 430 (CamW1) and a camera 431 (CamW2) viewing a target from different angles as do the two cameras in FIG. 6. Cameras CamW1 and CamW2 are responsive to a CamW1 wavelength range and a CamW2 wavelength range respectively. A second forward-looking camera system also located at the distal portion of the cannula comprises a camera CamF1 and a camera CamF2 viewing said target from different angles and responsive to a CamF1 wavelength range and a CamF2 wavelength range respectively, which ranges can be the same or overlap and comprise a selected narrow wavelength band light or fluorescence light. The CamW1 and CamW2 wavelength ranges are white light ranges and can be the same or overlapping. The CamF1 and CamF2 wavelength ranges can be a selected narrow wavelength band light range or fluorescence light range and can be the same or overlapping. Processing system 143 receives image data from said first and second camera systems and processes the received image data into a white light stereo image of the target and a selected narrow wavelength band image or fluorescent light image of the target overlaid in a composite image, and a display 150 displays said composite image. Display 150 can display any one or more of the white stereo image, the selected narrow wavelength band or fluorescence image, and the composite image. The positions of the cameras can be interchanged so long as the two cameras of the first camera system view the target from different angles and the two cameras of the second camera system also view the target from different angles. FIG. 14 also illustrates two channels, 414 and 416 in cannula 120 although a different number of channels can be used. FIG. 14 also illustrates respective light sources 242, 244, 432, 434, 433, 435, 437, and 439, for the four cameras, although a different number or a different arrangement of light sources can be used.

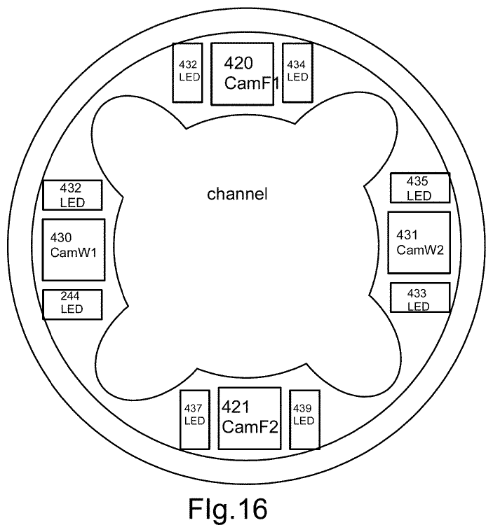

FIG. 16 is otherwise like FIG. 14 but illustrates a multi-camera, multispectral endoscope in which the four cameras and their light sources are arranged differently around a single channel 1502 in cannula 12.

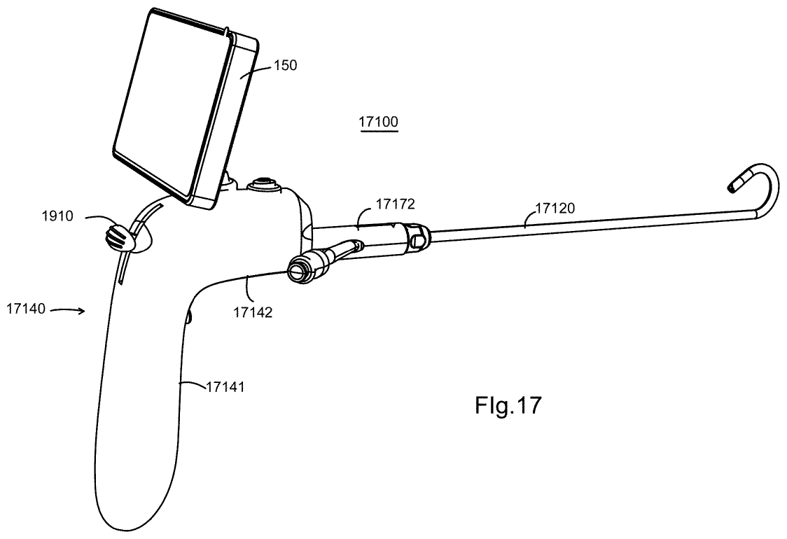

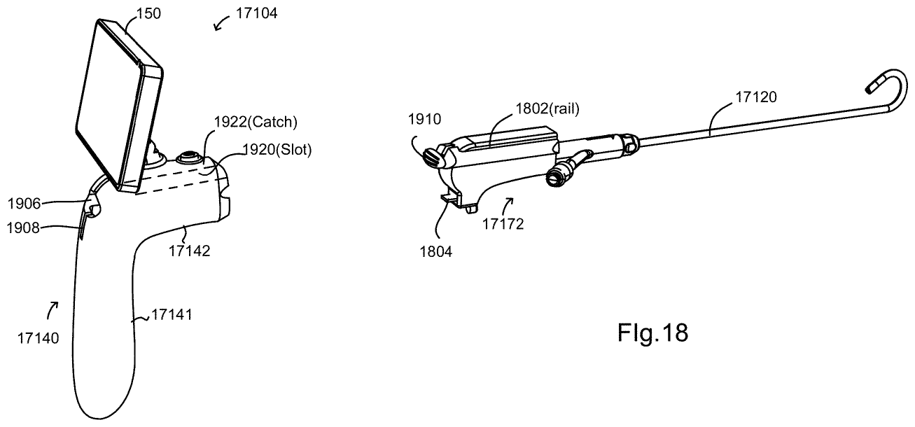

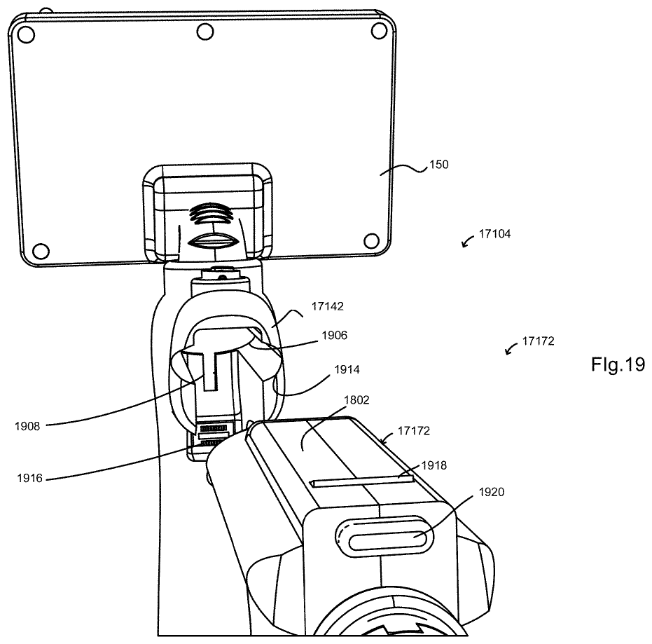

FIGS. 17-22 illustrate an endoscope t according to some embodiments. FIG. 17 is a perspective view of an assemble endoscope 17100 and FIG. 18 illustrates as separate units a portion 17104 that can be reusable and a portion 17102 that can be single-use, before they are removably assembled by sliding portion 1702 proximally into portion 17104. Portion 17104 comprises display 150 and an L-shaped handle portion 17140 formed of a downwardly extending handle 17141 configured to be grasped by a user's hand and an axially extending housing 17142. Display 150 is mounted on portion 17104. Portion 17102 comprises a hub 17172 that can be removably secured to housing 17142 and a cannula 17120 extends distally from the hub. Housing 17142 has an axially extending, downward facing slot 1902 (FIGS. 18 and 19) and hub 17172 comprises an axially extending, upwardly facing rail 1802 that is configured to slide into slot 1902 in the proximal direction and thereby removably secure portions 17102 and 17104 to each other. Hub 17172 has a proximally facing electrical connector 1804 (FIG. 18) at its proximal end and housing 17142 has a matching, distally facing electrical connector 1904. The two electrical connectors mate and make electrical contact when portions 17102 and 17104 are secured to each other to form the assembled endoscope 17100 that FIG. 17 illustrates. The proximal end of handle portion 17140 has an oval opening 1906 though which the proximal end of thumb lever passes and protrudes proximally when the endoscope is assembled to the form seen in FIG. 17. Oval opening 1906 connects to a vertical opening 1908 that allows a stem of thumb lever 1910 to move up and down. Thumb lever is a part of a bending mechanism, described below, that bends the distal end of cannula to the bent position seen in FIG. 17 and to any intermediate positions. The bend can be up or down.

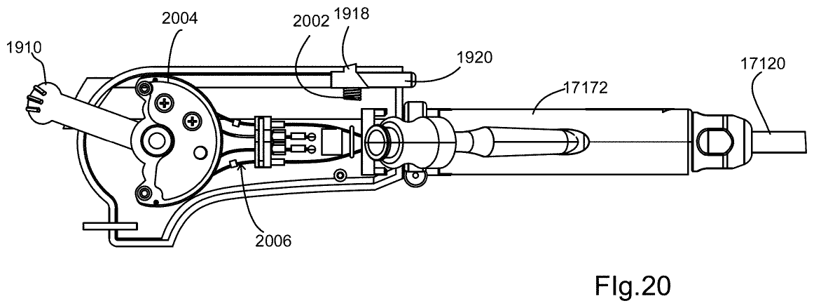

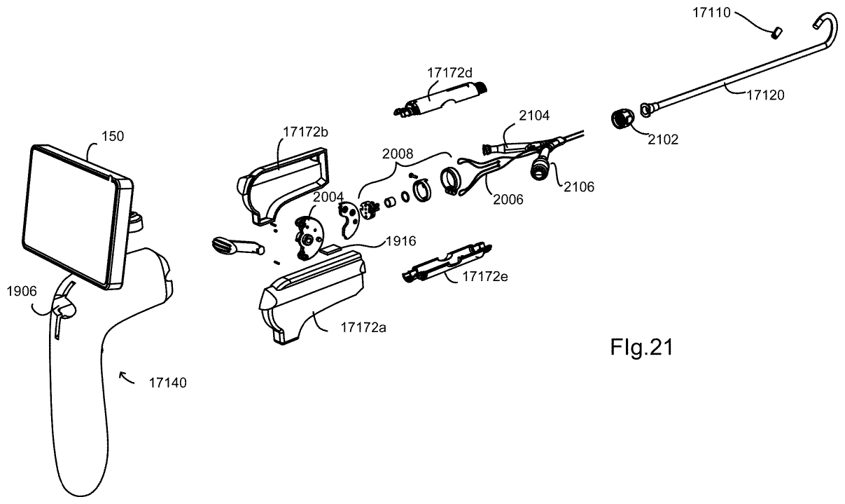

FIG. 19 illustrates in perspective portions of units 17102 and 17104. As seen from the distal end, unit 17104 has an opening 1912 in which portion 17102 slides. Seen in this opening is an axial, downwardly facing, C-shaped slot 1914 and an electrical connector 1916. As seen in FIG. 18, hub 17172 has an upwardly facing, axially extending rail 1802 that is T-shaped and configured to slide into slot 1914 when the endoscope is assembled. Also seen in FIG. 18 is an electrical connector 1804 that configured to mate and make electrical contact with electrical connector 1916 seen in FIG. 19 when the endoscope is assembled. FIG. 19 also shows a lock pin 1918 and a lock release 1920 that serve to securely lock portions 17102 and 17104 when the endoscope is assembled and are described in more detail below in connection with FIG. 20. Cannula 17120 has at its distal end a camera and lights module that can be any of the modules discussed above regarding other embodiments of the endoscope and connects to display 150 through internal cables and electrical connectors (in this case 1916 and 1804) as discussed above for other embodiments. Portion 17104 can have buttons or other manually operated inputs as discussed above for other embodiments of the endoscope to control the camera functions and/or other functions. Handle portion 17140 can house electronics for processing image data as discussed above for other embodiments. In some embodiments of endoscope 17100, display 150 can be eliminated and image data can be displayed at an external monitor connected wirelessly or through a cable with the camera module at the distal end of cannula 17120.

FIG. 20 is a sectional view of a portion of unit 17102 and shows lock pin 1918 that is urged up by a spring 2002 and a lock release 1920 that when pushed proximally pushes lock pin 1918 down and out of engagement with a catch 1922 (FIG. 18) that is a notch in the underside of opening 1914. When the endoscope is assembled, lock pin 1918 engages catch 1922 and holds units 17102 and 17104 together. After a medical procedure, the user pushes lock release 1920 to thereby release the engagement of pin 1918 and catch 1922 and pull unit 17102 distally out of unit 17104. FIG. 20 further illustrates a bending mechanism for bending the distal end of cannula 17120, which mechanism comprises a half-wheel 2004 mounted for rotation about its center and secured to thumb lever 1910 such that up-down motion of thumb lever 1910 translates to rotation of half-wheel 2004. Cables 2006 are secured to half-wheel 2006 and to the distal end of cannula 17120 such that rotation of half-wheel 2004 in one direction bends the distal end of the cannula in one direction and rotation of half-wheel 2004 in the opposite direction bends the distal end of the cannula in the opposite direction.

FIG. 21 is an exploded perspective view illustrating handle portion 17140 and display 150 and components of portion 17102. Hub 17172 is formed of right and left covers 17172a and 127172b and right and left covers 17172c and 17172d that extend distally therefrom. A cap 2102 screws ono the distal end of hub 17172 to affix cannula 17120 to hub 17172. Fluid ports 2104 and 2106 merge into a Luer fork that goes into cannula 17120, as do cables 2006. Electrical connector 1916 (which can be a DP20 connector) also is a part of unit 17102. Mechanical connection pieces 2008 help in assembling portion 17102 of the endoscope.

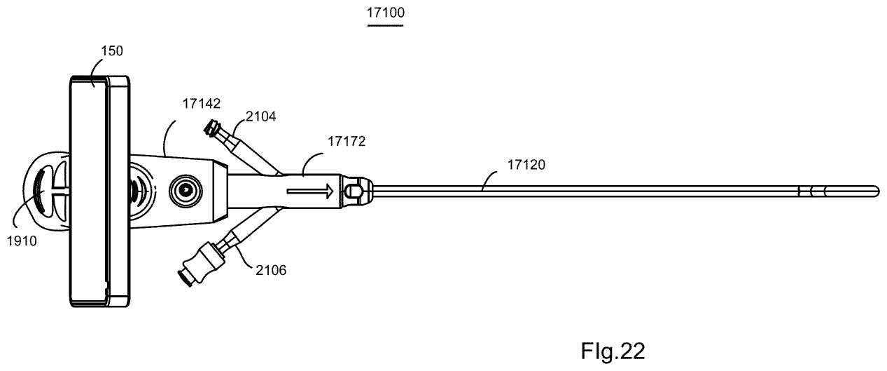

FIG. 22 is a top view of the assembled endoscope 17100 and illustrates the relative positions of the components, including fluid ports 2104 and 2106.

As noted above, features and components described in connection with one of the embodiments can be used in another of the described embodiments. As non-limiting examples, the different configurations of imaging and lighting modules can be used in any of the described endoscopes, the cannula bending mechanism described in connection with FIG. 20 can be used in any of the described endoscopes, etc.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

What it claimed is:

1. A multi-camera, multi-spectral endoscope comprising:
a cannula (120) configured for insertion in a patient;
a forward-looking camera CamW (430) at a distal portion of the cannula views a target and is responsive primarily to a wavelength range of white light;
an electrically controlled color filter (1202) also at the distal portion of the cannula and configured to selectively operate in a mode A to pass light primarily in a wavelength range of white light or in a mode B to pass to said camera CamF light primarily in a wavelength of selected narrow band or fluorescence light;
a forward-looking camera CamFA/B (12420) also at the distal portion of the cannula views said target from a different angle and through said color electrically controlled filter;
a processing system (143) configured to:
selectively switch said color filter between mode A and mode B, and
receive image data from said cameras CamW and CamFA/B and:
form a white light stereo image of the target when said filter is operating in mode A, but
form a selected narrow wavelength band image or a fluorescence light image from camera CamFA/B when said filter is operating in mode B; and
an image display (150);
wherein said processing system and image display are configured to form and display a composite image as an overlay of the white light stereo image and the selected narrow wavelength band image or fluorescent light image.

2. The multi-camera, multi-spectral endoscope of claim 1, further including a fluid hub (172) from which said cannula extends distally and a hand piece (140) to which the fluid hub us secured.

3. The multi-camera, multi-spectral endoscope of claim 2, in which the fluid hub and cannula comprise a single-use unit (102) and said hand piece comprises a reusable unit (104) and is releasably secured to the single-use unit.

4. The multi-camera, multi-spectral endoscope of claim 3, in which the single-use unit extends along a longitudinal axis, the reusable unit has an upper portion that has an open slot extending along said longitudinal axis and a handle portion extending along a handle axis transverse to the longitudinal axis, wherein said fluid hub is configured to releasably snap into said open slot.

5. The multi-camera, multi-spectral endoscope of claim 3, wherein the reusable unit includes a manual bend controller mounted at a proximal end thereof and said single use unit includes a bending mechanism that automatically engages said manual bend controller when the single-use unit is snapped into said slot and responds to manual operation of the bend controller to selectively bend the distal portion of the cannula.

6. The multi-camera, multi-spectral endoscope of claim 2, in which said cannula is configured to rotate relative to a proximal portion of said fluid hub.

7. The multi-camera, multi-spectral endoscope of claim 1, further including a manual bend controller and wherein said cannula's distal portion is configured to bend in response to operation of said manual bend control.

8. The multi-camera, multi-spectral endoscope of claim 1, in which said camera CamF has a lower spatial resolution than said camera CamW at least when said filter is operating in said mode B.

9. A multi-camera, multi-spectral endoscope comprising:
a tubular cannula (120) configured for insertion in a patient;
a first forward-looking camera system located at a distal portion of the cannula and comprising two cameras CamW1 and CamW2 viewing the same target from different angles and responsive primarily to a CamW1 wavelength range and a CamW2 wavelength range respectively;
a second camera system located at the distal portion of the cannula and comprising a camera CamF that also views said target but is responsive primarily to a CamF wavelength range that is different from at least one of the CamW1 and CamW2 wavelength ranges;
a processing system configured to receive image data from said first and second camera systems and to process the received image data into a stereo image of the target using image data from CamW1 and Cam W2, a two-dimensional (2D) image of the target using image data from Cam F, and a composite image of the target overlaying said stereo and said 2D images; and
a display configured to display said composite image.

10. The multi-camera, multi-spectral endoscope of claim 7, in which said wavelength ranges CamW1 and CamW2 overlap.

11. The multi-camera, multi-spectral endoscope of claim 7, in which said wavelength ranges CamW1 and CamW2 are white light ranges.

12. The multi-camera, multi-spectral endoscope of claim 9, in which said CamF range is a selected narrow wavelength range or fluorescence light.

13. The multi-camera, multi-spectral endoscope of claim 9, wherein said 2D image represents target areas that emit fluoresce above a threshold of likely abnormal tissue, thereby highlighting likely abnormal tissue in said composite image.

14. The multi-camera, multi-spectral endoscope of claim 11, in which said composite image comprises an overlay in which said 2D image is visible in areas of said 2D image.

15. The multi-camera, multi-spectral endoscope of claim 7, in which said camera CamF has a lower spatial resolution than at least one of said cameras CamW1 and CamW2.

16. The multi-camera, multi-spectral endoscope of claim 7, further including at least one internal channel (414, 416) in said in which said cannula, a fluid hub from which said cannula extends distally and which communicates with said internal channel, wherein said cannula is configured to rotate relative to a proximal portion of said fluid hub.

17. The multi-camera, multi-spectral endoscope of claim 14, further including a hand piece (104) to which said fluid hub releasably attaches and which houses at least a portion of said processing system.

18. The multi-camera, multi-spectral endoscope of claim 15, in which said display is mounted on said hand piece.

19. The multi-camera, multi-spectral endoscope of claim 7, further including a manual bend controller and wherein said cannula's distal portion is configured to bend in response to operation of said manual bend control.

20. A multi-camera, multi-spectral endoscope comprising:
a cannula (120) configured for insertion in a patient;
a first forward-looking camera system at a distal portion of the cannula and comprising a camera CamW1 and a camera CamW2 viewing a target from different angles and responsive primarily to a CamW1 wavelength range and a CamW2 wavelength range respectively;
a second forward-looking camera system also located at the distal portion of the cannula and comprising a camera CamF1 and a camera CamF2 viewing said target from different angles and responsive primarily to a CamF1 wavelength range and a CamF2 wavelength range respectively that differ from at least one of said CamW1 and CamW wavelengths;
a processing system receiving image data from said first and second camera systems and processing the received image data into a CamW image of the target based on the image data from said cameras CamW1 and CamW2 and a CamF images of the target based on image data from said cameras CamF1 and CamF2 overlaid in a composite image; and
a display configured to displays said composite image.

21. The multi-camera, multi-spectral endoscope of claim 12, in which said CamW1 and CamW2 wavelength ranges are white light ranges and said CamF1 and CamF2 wavelength ranges are a selected narrow wavelength band range or a fluorescence light range.

22. The multi-camera, multi-spectral endoscope of claim 12, in which each of said images CamW and CamF is a stereo image of the target, and said composite image is an overlay in which the images CamW and CamF are spatially registered.

23. An endoscope comprising:
an L-shaped handle portion comprising a downwardly extending handle and an axially extending housing;
a hub removably secured to a proximal end of the housing and a cannula extending distally from the hub;
wherein:
one of said housing and hub comprises an axially extending slot that faces down and the other comprises an axially extending rail that faces up and is configured to slide into the slot in the proximal direction and thereby removably secure the hub and cannula to the handle portion;
said hub and said housing comprises respective electrical connectors that mate and make electrical contact when the housing and hub are secured to each other;
said proximal portion of the handle portion comprises an opening and said hub and cannula comprise a bending mechanism that is configured to bend a distal portion of the cannula and includes a proximally extending thumb lever that passes through said opening and protrudes distally from the handle portion when the hub and handle portion are secured to each other and manual action on said thumb lever controls bending of said distal portion of the cannula;
a camera module at the distal portion of the cannula; and
a display operatively coupled with the camera module to receive image data therefrom and display images based thereon.

24. The endoscope of claim 23, in which the bending mechanism comprises a wheel mounted in said housing for rotation and coupled with said bending lever to rotate in response to manipulation of the bending lever and cables coupled with the wheel and to the distant portion of the cannula to translate rotation of the wheel to bending of said distal portion of the cannula.

25. The endoscope of claim 23 in which said hub and cannula separate from the handle portion by manual sliding of the hub in the distal direction relative to the handle portion.

26. The endoscope of claim 23 including a lock pin in one of the housing and hub and a catch in the other, configured to engage when the endoscope is assembled and hold the hub to the housing, and a manually operated release to disengage the lock pin and catch from each other to thereby allow removal of the hub from the housing.
